(19) United States
(12) Patent Application Publication
(10) Pub. No.: US 2017/0071778 A1
(43) Pub. Date: Mar. 16, 2017

(54) HEAD SUPPORT SYSTEM

(71) Applicant: ABLE LOGIC SYSTEMS LIMITED, Essex (GB)

(72) Inventors: Malcolm Twitchin, Essex (GB); Adrian Clark, Essex (GB)

(73) Assignee: ABLE LOGIC SYSTEMS LIMITED, Essex (GB)

(21) Appl. No.: 15/122,385

(22) PCT Filed: Feb. 27, 2015
(86) PCT No.: PCT/GB2015/050582

§ 371 Date: Aug. 29, 2016

(30) Foreign Application Priority Data
Feb. 28, 2014 (GB) 1403546.3

(51) Int. Cl. A61F 5/37; A61G 13/12; A61G 1/04

(52) U.S. Cl. CPC A61F 5/3707; A61G 1/04; A61G 13/121

(57) ABSTRACT

The invention relates to a head support system for a stretcher. The invention provides a base support (1) and head block (11) which can be applied to a stretcher in order to restrict movement of a patient's head.

The invention relates to a head support (or immobiliser) system for a stretcher. More specifically, the invention relates to a base support, head block and sets thereof.

As is comprehensively appreciated in the medical community, it can be of profound importance that following, for example, a pre-hospital traumatic spinal head and neck injury incident such as a traumatic sporting accident, a subject's head is moved as little as possible prior to a fuller medical assessment being conducted at a care facility. This practice is adopted in order to try and minimise the chances of any secondary head or spinal injury being caused or exacerbated while a subject is being transported to a definitive care facility.

In accordance with this practice it is widely appreciated that it is desirable to immobilise a patient's head when they are being stretchered by emergency personnel. Consequently, head immobiliser systems which are specifically applied to and used with stretchers have been developed. Such head immobilisers encompass two blocks for restricting rotation and sideways tilting of a subject's head (head blocks) and a complementary base support to which the blocks can be attached.

By their nature, however, existing head immobiliser systems are awkward and problematic to use. Trying to fit a head immobiliser when a patient is on a stretcher, for example, may be detrimental (e.g. due to undue movement of the patient both from moving a support underneath a patient and securing clips). To fit such systems can therefore mean awkward, careful manipulation while a patient is in situ.

If existing head immobiliser parts are first applied to the stretcher, however, e.g. the base support is pre-attached, it can interfere with patient transfer to the stretcher by hindering certain movements and positions. Indeed, it may simply not practically be feasible to pre-apply a base unit. This is particularly an issue in instances where a two-part, ‘scoop’ stretcher is used.

In this regard, a scoop stretcher (also called a Robertson orthopaedic or clamshell stretcher) is one which can be split into sections that can be manoeuvred either side of a patient then brought together under the patient and secured to each other. As will be understood by a person skilled in the art, it may not be necessary for the divided sections of the stretcher to completely separate from each other, though this is often how they are used/function in practice. Their use is particularly desirable where spinal injury may be a concern because they reduce the amount of patient movement necessary for patient placement on the stretcher and lower chances of undesirable movement. Accordingly, they are frequently used in conjunction with head immobilisers during patient transfer.

Moreover, existing head immobiliser systems have a propensity to move on various types of stretcher, their base supports frequently shifting or slipping over the surface, e.g. as a consequence of force or jarring.

In situations, such as those described above, where the base plates of immobilisers are problematic to deploy, in order to facilitate patient transfer medical practitioners frequently opt to improvise head immobilisation. For example, existing head blocks (designed to sit either side of a patient's head) may be used upside down (with respect to their intended orientation) and secured together using adhesive tape. Alternatively, IV fluid bags, rolled up towels or blankets may be used. Such improvised systems are clearly not ideal, however, and, moreover, it can be difficult to get tape to suitably adhere for appropriate head restraint, particularly in wet weather and/or when the subject may be perspiring (e.g. if an incident occurs during a sporting event).

It is also a consideration that stretchers come in a vast range of different shapes and sizes. Dimensions and features vary. In some, for example, the stretcher base, upon which a patient lies, is flat; in others it has sloped sides. Accordingly, components of head immobiliser systems are often unsuitable for application to different stretchers.

As can be seen, therefore, existing head immobilisers are not without disadvantages and so the present invention aims to address such problems associated with the prior art.

According to an aspect of the present invention there is provided a base support for a head immobiliser system for use with a stretcher, the base support being operable to be attached to one side of a stretcher such that, in use, a head block for supporting a person's head can be placed on it;

wherein the base support comprises a rigid plate, at least one head restraining strap connector secured to and extending from the plate for connection to a head restraining strap, and at least one attachment strap secured to and extending from the plate, for attaching the base support to a stretcher, and comprising an area of a component of a hook and loop fastener;

wherein the plate comprises an area of a component of a hook and loop fastener, on its upper surface, for attachment to a complementary section of fastener component on a head block surface, and an area of a component of a hook and loop fastener for attachment to the fastener component on the at least one attachment strap;

and wherein one side of the rigid plate has an extension operable to be fitted at least partially around the edge of a stretcher.

As will be understood, the present invention is intended to be used as one of a pair of base supports, with one being used each side of a stretcher next to patient's head. The present invention thus allows for a much more desirable approach to head immobilisation.

For example, the thin, plate-like and relatively rigid nature of the base support is very beneficial because, for example, in comparison to systems of the prior art it is much less bulky. In this regard, head immobilisers currently on the market have base supports made principally of a foam-type material such as polyurethane. As a consequence they tend to be somewhat bulky and flexible and hence may not be easily moved under a person's head, requiring the head to be lifted and thus risking spinal damage.

The invention may also be pre-applied to a stretcher in advance of a person being transferred, and in such circumstances the relatively thin and rigid nature may be less cumbersome than prior art base supports with respect to patient transfer.

The plate extension also makes the support, in general, much easier to fit and more secure, by giving clear placement guidance and resistance to lateral movement.

Furthermore, not only can the base support be more easily put into position (even when a patient is on the stretcher), a stretcher does not need to be lifted in order to attach the plates because the attachment strap (or straps) can loop around/through a handle of the stretcher, for example, and then be easily secured to the base plate (by virtue of the hook/loop material). The securing of an attachment strap (and hence base support) is thus a quick, straightforward and convenient operation.

In this regard, most stretchers currently in use have a series of gaps around the edge to facilitate gripping the stretcher along its length and at the ends.

Hook and loop fasteners allow for reversible yet appropriately secure attachment.

Though it will be understood that the invention may be used with standard (non-separating) stretchers, there may be particular benefits with respect to scoop stretchers. Specifically, for example, one of each of the base supports can be pre-attached to respective sides of the scoop stretcher and thus, when the patient is placed on the stretcher the base supports are already in place, mitigating the need for further manipulating the patient in order to fit a head immobiliser. This differs significantly from prior art systems which by, for example, having a single piece base unit, may not practically be pre-attached to a scoop stretcher. Accordingly, the invention is suitable for pre-attachment to a scoop stretcher.

Moreover, even if fitted after a person is on a stretcher, the two-piece factor of the invention could minimise/negate patient movement irrespective of the stretcher type.

As mentioned above, one side of the rigid plate extends in a manner (e.g. into/through different planes) such that it can be fitted at least partially around the edge of a stretcher. For example, one side of the plate may extend generally at a right angle or may curve into a half-pipe or quarter-pipe, for example, in/adjacent to which the edge of a stretcher can sit. It will be understood that the extension (flange) may be straight or rounded, e.g. different stretchers may be better served by a right angled or curved side to the plate (depending on whether the stretcher edge is generally straight or rounded). The support plate extension can assist with positively locating the base support on a stretcher and making the attachment of the base support to a stretcher more secure (by, for example, resisting movement if pulled).

It will be understood that the stretcher edge around/adjacent to which the extension is intended to sit is a left/right side of the stretcher near the patient's head.

Conveniently the extension does not, in use, extend beyond the bottom of the stretcher. Accordingly, in view of the dimensions of most current stretchers, the side preferably extends for (approximately) 5 mm to 40 mm, more preferably 10 mm to 40 mm, more preferably 15 mm to 35 mm, more preferably 20 mm to 30 mm, more preferably 22 mm to 27 mm, more preferably 24 mm or 25 mm below the plane of the plate.

Another option would be for the side to extend below the bottom of the stretcher but also to curve or extend around under the stretcher. The preference would likely be for closer but not too tight fitting between the base support and the stretcher side and bottom.

The improved functionality of the present invention thus significantly militates against unduly moving a patient and using makeshift and impractical immobilisation methods.

The rigid support plate may comprise any material or materials that could practically be used to make a relatively thin, rigid and generally flat component. It may, for example, comprise a framework (the underlying part/structure on which fastener component(s) is/are attached) that comprises, or is made of, fibre glass, carbon fibre, moulded plastic, metal (e.g. aluminium) and/or an alloy such as stainless steel.

Conveniently the framework is a metal plate. Preferably the metal is aluminium.

Conveniently hook/loop fastener component is attached directly to the framework upper surface, though there could be intervening elements.

Conveniently the aluminium or other framework material is sandblasted in order to facilitate attachment of fastener component. Sandblasting is not compulsory, however. Conveniently an adhesive (e.g. glue) is used to attach fastener component to the framework material.

Preferably the rigid support plate is thin enough and made of materials such that the base support does not interfere with X-rays (i.e. may be considered translucent to X-rays) and so may be left in situ while an X-ray image of a patient is taken. In this regard, it has been found that using an aluminium metal plate of 2 mm thickness (with hook/loop fastener thereon) is translucent to X-rays.

It will be apparent that, to be suitable for attachment to a head block, the area of fastener component for head block attachment should be on the plate surface which, in use, would be its upper surface. Further, for greater convenience the area of fastener component for attachment strap attachment is also on the plate upper surface (and, as discussed further below, for greatest convenience it is the same area as for the head block). An attachment strap could otherwise be attached to the plate underside.

The rigid plate may comprise more than one fastener component for head block and attachment strap attachment (e.g. an area of hook and an area of loop). Preferably, however, the fastener component on the rigid plate for attachment to the head block and the fastener component on the rigid plate for attachment to the attachment strap are the same, e.g. one component can serve to adhere both to a head block and the base support attachment strap (i.e. both the head block and strap separately comprise the same component).

The areas of fastener component for head block and attachment strap attachment may be, but need not be, physically discrete. Accordingly, they may be thought of as continuous or the same area (e.g. one area may serve both functions). In this regard, it will be apparent that placement of attachment straps and head blocks may vary somewhat between uses. Furthermore, as discussed further below, an attachment strap itself may further comprise the second hook and loop fastener component for adhering a head block.

It will also be understood that any area of fastener component (whether it can serve one or multiple purposes) may comprise discrete sections, for example a plurality of patches to one or more of which a component or components (e.g. head block and/or attachment strap) may be adhered. Multiple strips or discs, for example, could be considered to correspond to an area of fastener component. A component being attached clearly may not necessarily need to contact all sections, this may depend on circumstances.

Conveniently substantially all of the upper surface of the rigid plate is covered in fastener component. In this way, there may be greater flexibility for attachment strap and head block placement.

Preferably the rigid support plate comprises a layer of material, e.g. textile, on at least a portion of its underside for protecting a stretcher when the base support is in use. The material may be a hook/loop fastener component. Preferably (particularly when there is only one hook/loop fastener component on the upper surface) the material is the same as fastener component that is on the upper surface, in which case the upper surface material may conveniently continue round the edge of the plate to the underside. In this regard, the underside of the plate is taken to be that side which, in use, would be adjacent to a stretcher and not used for adhering a head block. In this way the stretcher is protected against wear and tear from the plate and an optional attachment point for a head pad (and, as discussed above, the attachment strap) is provided. In this regard, a head pad may, if desired, be placed on a stretcher between two supports of the invention in order to provide a platform on which a patient's head can rest, the head pad having straps for base support attachment. This option may, as discussed further below, depend on the support plate dimensions. A head pad could also be attached to the upper surface of a base support.

In line with discussion above, there may be more than one section of material (e.g. fastener component) on the underside. Material/fastener component may cover most, optionally (substantially) all, of the underside of the base support plate.

One or more alternatives to fastener component may otherwise or also be applied to the underside in order to provide protection for the stretcher.

Preferably loop fastener component is used on the support plate for any one or more of the areas of fastener material as may be relevant. This is particularly relevant with respect to the upper surface area for attaching head blocks. In this way existing head blocks (which have hook component) may be used in combination with the present invention. Preferably all fastener component on the base support is loop component.

Fastener (hook/loop) component (and other material as relevant) may conveniently be secured to the support plate framework by adhesive. Conveniently the hook and loop fastener is Velcro®.

Conveniently the support plate is (generally) rectangular in shape. For a (generally) rectangular support plate, conveniently one of the long edges extends in a manner for fitting around/adjacent to the edge of a stretcher as described above.

Preferably there is a plurality of head restraining strap connectors attached to the base support plate. In this way multiple head restraining straps may be used to secure head blocks and/or different head strap positioning may be more easily selected.

Preferably there are two, three, four or five head restraining strap connectors. Conveniently they extend from the same side of the support plate.

Preferably the one or more head restraining strap connector(s) is/are secured to what would be, in use, the upper side of the support plate.

Preferably the one or more head strap connector(s) extend(s) from the base plate extension/flange (which, in use, runs along the outside edge of the stretcher).

Conveniently the one or more of the head restraining strap connector(s) each comprise a D-ring for attachment to a head restraining strap.

Preferably there is a plurality of attachment straps attached to the base support plate. In this way the base support may be more securely fastened to a stretcher. In this regard, stretchers generally in use today have multiple hand grips through which such straps can easily be fed and subsequently secured.

Preferably, e.g. when the base support is generally rectangular, the one or more attachment strap(s) extend(s) from a side or sides of the base plate which run(s) (generally) perpendicular to the side with the extension.

Conveniently the one or more attachment strap(s) is/are secured to what would be, in use, the top side of the plate.

Conveniently there are two attachment straps. Preferably they extend from opposite ends of the base support plate.

The one or more head strap connector(s) and/or the one or more attachment strap(s) may comprise webbing.

Preferably the fastener component area on the one or more attachment strap(s) comprises back to back lengths of hook and loop components (one component on either face). Preferably they are stitched together. Webbing may be incorporated between the two materials for added strength. Having both hook and loop material on the attachment straps means that, when adhered to the upper surface of the base plate, the other side of the strap can act as part of the attachment surface for a head block. Should there be only one fastener component on an attachment strap it is preferably hook component, however.

Conveniently the fastener component area on the one or more attachment strap(s) is at or near the distal end of the strap(s). In this regard, the distal end is taken to be the end which can be at a distance from the rigid plate, e.g. when the base support is not in use. As another option, the fastener component area may extend for all or substantially all of the length of the attachment strap. Accordingly, the whole length of the strap may comprise back to back lengths of hook and loop components as discussed above.

Methods for securing the one or more head restraining strap connector(s) and/or the one or more attachment strap(s) may include using plastic or metal screws, rivets or nuts and bolts etc., e.g. as the basis for stud fastenings. For example, holes may be drilled, lasered or stamped in the framework material (e.g. aluminium) and straps secured by being held by a rivet passing through the strap and the framework.

Another option may be to pass straps through slits in the rigid plate and secure them by stitching them back onto themselves.

If hook/loop fastener is first applied to the framework then fastenings may also function to further secure such material.

Preferably the support plate framework is (approximately) 0.1 mm-10 mm thick, more preferably 0.5 mm-5 mm, more preferably 1 mm-3 mm thick. More preferably it is (approximately) 2 mm thick. In this way it may not unduly interfere with X-rays.

The plate dimensions may vary, e.g. in consideration of the different stretchers with which they may be used. In this regard, the strap (head strap connectors and/or attachment straps) positions may also vary depending on stretcher type. It will also be apparent that the number of connectors or straps may conveniently vary with respect to different stretchers.

Preferably the plate length (the length of the extended/flanged side and that opposite to it) is (approximately) 50 mm to 350 mm, more preferably 50 mm to 300 mm, more preferably 100 mm to 280 mm, more preferably 150 mm to 250 mm, more preferably 180 mm to 220 mm, more preferably 190 mm to 210 mm. Conveniently the plate length is (approximately) 190 mm, 200 mm, 210 mm, 290 mm, 300 mm or 310 mm.

Preferably the plate width (the length of the sides that run perpendicular to the extended/flanged side) is (approximately) 50 mm to 250 mm, more preferably 100 mm to 200 mm, more preferably 130 mm to 190 mm, more preferably 150 mm to 170 mm. Conveniently the plate width is (approximately) 160 mm or 210 mm. The width relates to the distance between the perimeter edges and thus may be considered to extend to a line that extends upward from the outside edge of the curved side.

In this regard, it is preferable that when a pair of the base supports are applied to a stretcher (one either side), there would remain a gap between them. It is in the gap that a person's head would principally, or completely, rest. Accordingly, the plate width is conveniently such that it corresponds to less than half the stretcher width at the head end. The optimal dimensions may depend on the stretcher to be used. As discussed elsewhere, a head pad may optionally be used between two base supports.

The plate dimensions may be approximately 160 mm (width) x 190 mm (length), e.g. +/− 15%. Conveniently, the plate dimensions are 160 mm×190 mm or 210 mm for some stretchers. For other stretchers, such as military stretchers, dimensions may preferably be about 215 mm×300 mm.

As another aid to stretcher attachment a base support may conveniently have a hole, e.g. a slit/slot, through which an attachment strap could be passed. In this way more convenient attachment of the support to a stretcher may be enabled by facilitating passage of a strap from one side of the support to the other. Further, a base support could have more than one such hole.

In addition to the features discussed above, a base support of the present invention may, if desired, be otherwise designed, shaped or modified so that it is more convenient to use with a particular stretcher. For example, particular dimensions may be chosen and corners or edges may be trimmed, rounded, ridged or angled to better fit contours of different stretchers.

According to another aspect of the present invention there is provided a set of head immobiliser base supports for use with a stretcher, the set comprising a pair of the support bases disclosed herein for use on opposite sides of a stretcher.

Optionally, the support bases may each be specifically adapted for a different side of the stretcher (e.g. as discussed above with respect to base supports being designed to specifically accommodate different stretcher features). In this way, a set of base supports may be considered left and right “handed”. Otherwise, a base support may be used on either side of a stretcher.

Optionally the stretcher is a scoop stretcher.

According to another aspect of the present invention there is provided a head block for use with a stretcher head immobiliser system, such that, in use, a person's head may be supported against a side of the head block, the head block comprising a base on at least a portion of the surface of which there is a layer of one component of a hook and loop fastener for attachment of the head block to a base support of a head immobiliser, wherein the base is rounded in a convex manner such that the head block may be used with a range of stretchers.

The head block may be applied directly to a suitable stretcher but is conveniently applied to a base support.

Preferably the base curves steadily upwards from the bottom of the side against which a person's head may be supported. Preferably the gradient of the base (i.e. of tangents to the base when the side against which a person's head may be supported is considered vertical) steadily increases from approximately 0° as the base curves upwards from the side against which a person's head may be supported.

The curved bottom of the head block may be thought of as an arc. The arc may be a circular arc or an elliptical arc. Conveniently the arc is an elliptical arc. Preferably the arc spans a vertical distance of (approximately) 10 mm to 40 mm, more preferably 15 mm to 35 mm, more preferably 20 mm to 30 mm. Preferably the arc spans a vertical distance of (approximately) 25 mm. (The vertical distance relates to the upwards displacement when the block is in its natural in-use orientation, though the actual displacement in practice may vary, e.g. depending on the stretcher being used, as discussed further below.)

Preferably the arc spans a horizontal distance of (approximately) 70 mm to 200 mm, more preferably 80 mm to 160 mm, more preferably 90 mm to 140 mm, more preferably 100 mm to 120 mm. Preferably the arc spans a horizontal distance of (approximately) 110 mm.

Example head block dimensions are height 140 mm, length 240 mm (meaning the dimension which, in use, would extend at least partially between a person's chin and the top of his/her head) and top width (which may be narrower than the base) 50 mm. It will be apparent, however, that there may be significant variation of head block shapes and dimensions.

Conveniently the side adjacent which a person's head may rest forms a right angle with the top of the block.

The outward rounding of the base of the head block, being the part which contacts the support plate/stretcher, means that the head block can be securely applied to a stretcher having a sloping base while the side of the block which rests beside a person's head remains substantially vertical. For flat-bottomed blocks either they would have to be especially designed for each stretcher or, in use, the side of a standard block can end up sharply angled towards a person's head.

A curve is beneficial over a sloping base because it gives greater security/versatility, e.g. with respect to stretchers having sloped sides of different gradients and different base supports. For example, a greater or lesser proportion of the base of the head block may be in contact with the base support/stretcher depending on the gradient of the stretcher bottom.

The head block may also be used with standard (non-sloped) stretchers.

Conveniently (substantially) all of the base has a layer of one component of a hook and loop fastener. In this way, there may be greater flexibility and versatility for attaching the head block.

As discussed above, it will be understood that an area of fastener component may comprise discrete sections, for example a plurality of patches for adhering a head block. Multiple strips or discs, for example, could make up an area of fastener component. Clearly it may be that not all sections need necessarily be used to adhere the head block, this may depend on circumstances.

Preferably the component of the hook and loop fastener is the hook component/material. In this way the head block is in line with convention. Accordingly, the head block may be used with existing base supports of the prior art.

It will be appreciated that the principle of a curved base may be applied to head blocks whose features are otherwise known in the art. Generally, such head blocks may be thought of as being brick-like in nature. Frequently, for example, head blocks generally take the form of a quadrilateral prism and, in use, it is one of the longest sides which sits beside a person's head. They may or may not have a cut out portion (at the top) to facilitate head strap use and/or may or may not have a hole extending between the sides (to help accommodate a patient's ear, for comfort, hearing and inspection purposes and also to facilitate gripping). The side against which a person's head may be supported is generally vertical (though other formats are known), the opposite side can be sloped. The prism end may optionally be rectangular or have acute and obtuse angles (often it will have at least two right angles). They may be made of polyurethane foam.

It will be understood that these example features are given for general illustration purposes and other quite different examples of head blocks are also known in the art.

Scoop stretchers are more likely to have a sloped base and thus this aspect of the invention may be particularly beneficial with respect to such a stretcher.

According to another aspect of the present invention there is provided a head pad for use between two base supports of the invention, the head pad comprising a padded area on which a person's head may rest and two attachment straps, one each extending from opposite sides of the padded area, comprising a component of hook and loop fastener for attaching the head pad to the base supports.

According to another aspect of the present invention there is provided a head immobiliser system for use with a stretcher, the said system comprising base supports and head blocks, wherein the base supports and/or the head blocks are as disclosed herein according to aspects of the invention. In use, two head blocks may each be adhered to a base support such that a subject's head may be supported therebetween.

Preferably the system components are all as disclosed herein in accordance with the invention. As indicated previously, however, base supports and head blocks of the invention may also be used with existing prior art immobiliser components.

Optionally the stretcher is a scoop stretcher.

Head pads of the invention, to sit underneath a person's head, may also be used and supplied with any other part of the present invention.

The present invention will now be described with reference to the accompanying drawings, of which:

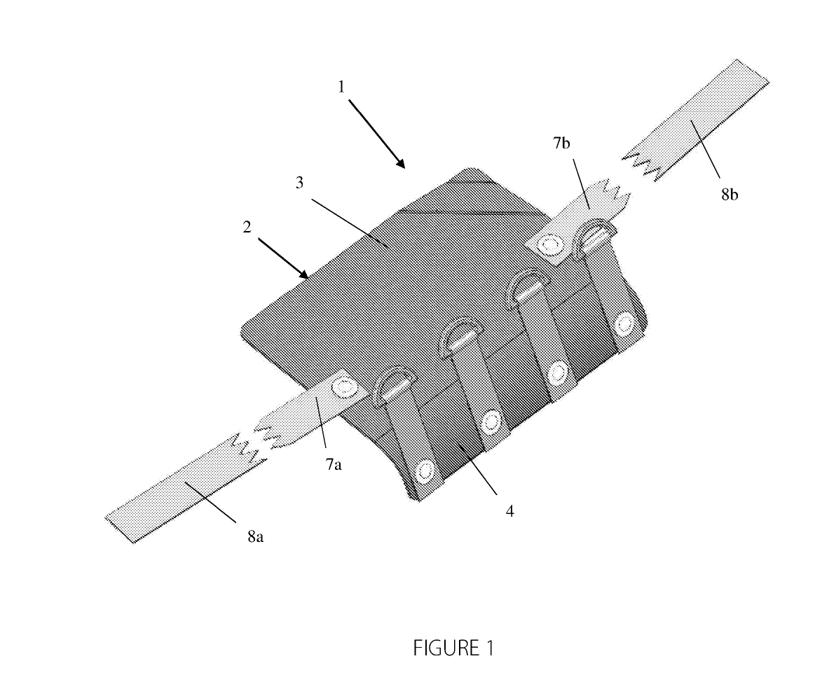

FIG. 1 shows the upper side of a base support of the present invention.

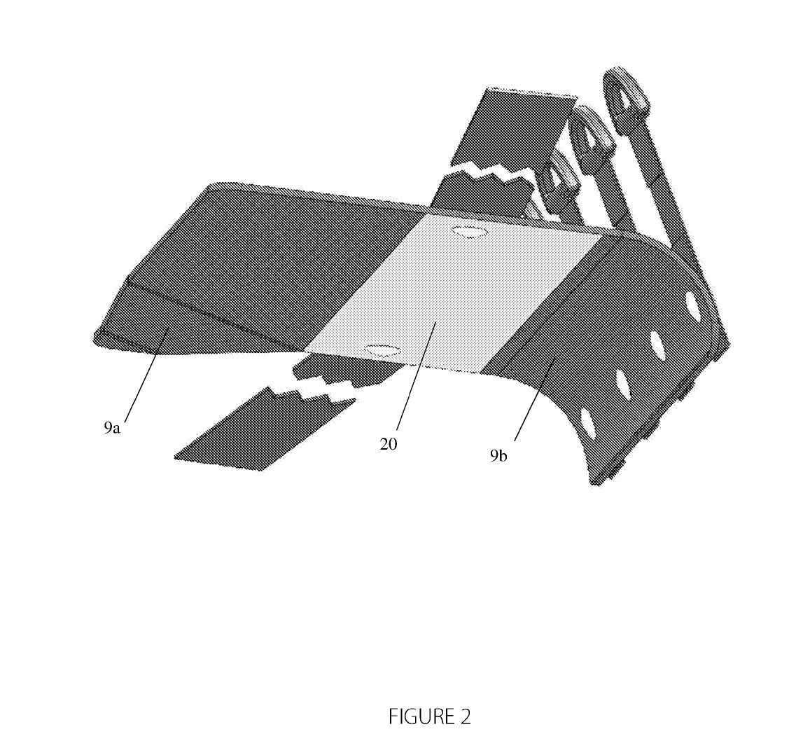

FIG. 2 shows the underside of a base support as in FIG. 1.

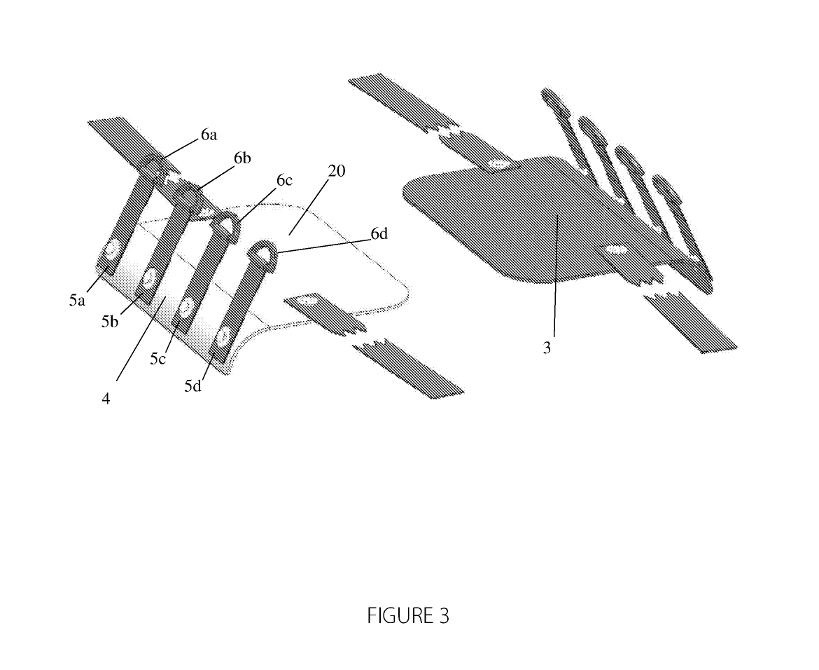

FIG. 3 shows the upper side of a pair of base supports of the invention. One of them is shown without an upper side layer of hook/loop fastener for illustration purposes.

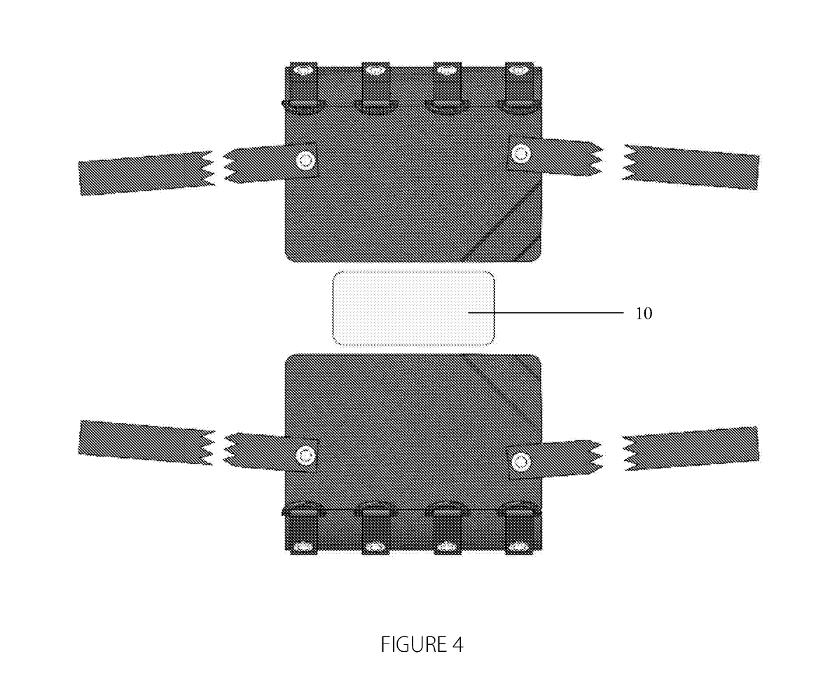

FIG. 4 shows two base supports of the invention with a head pad between them from above.

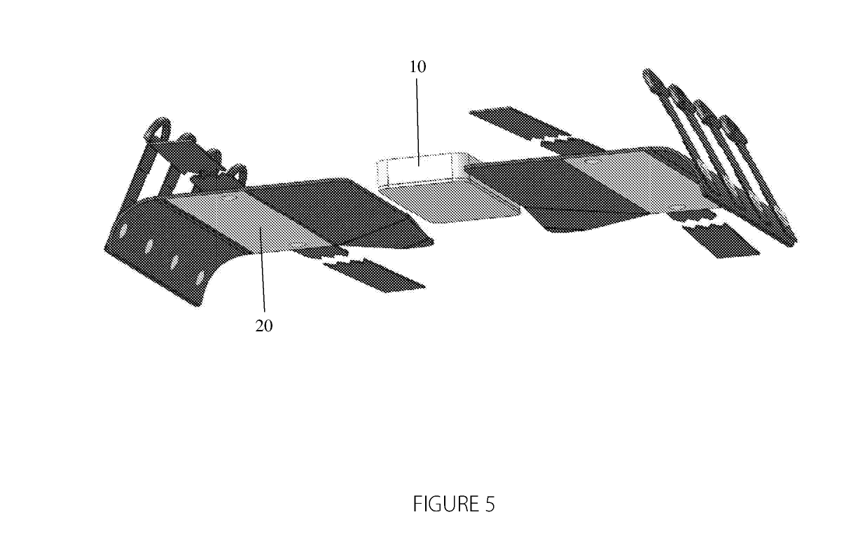

FIG. 5 shows two base supports of the invention and a head pad between them from below.

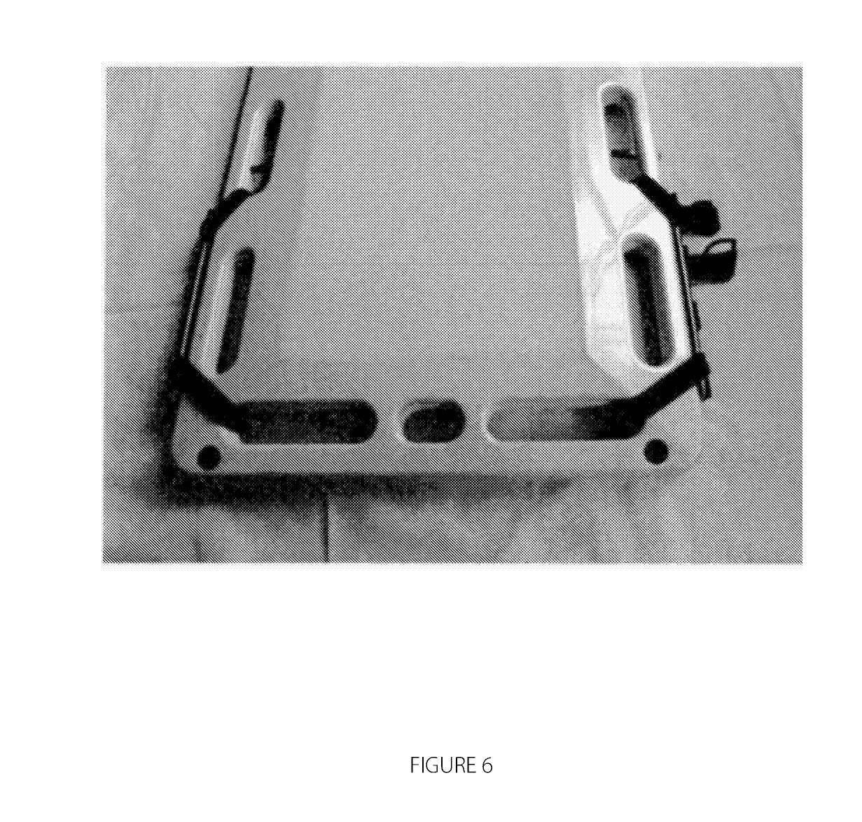

FIG. 6 shows the underside of a stretcher with two base supports of the invention attached.

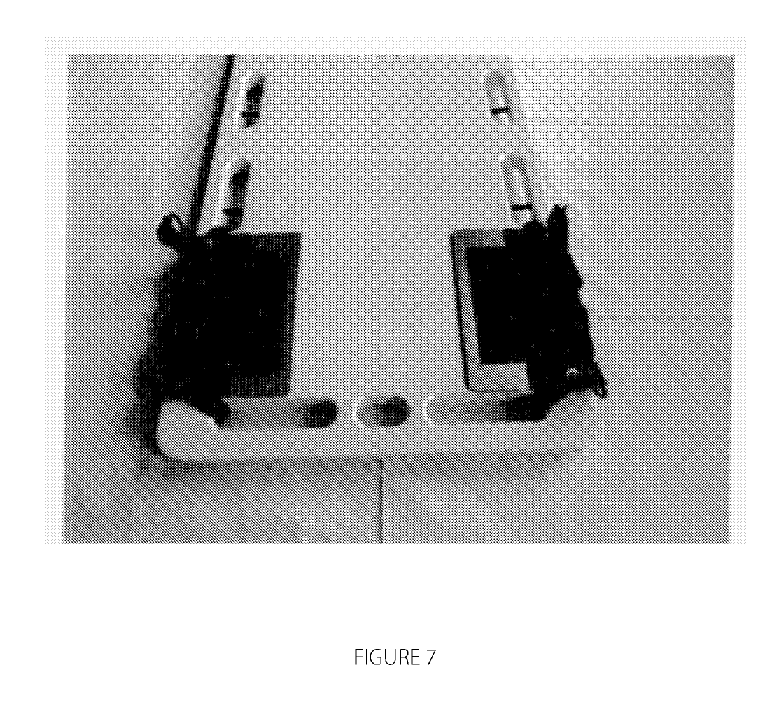

FIG. 7 shows the upper side of the stretcher of FIG. 6 with two base supports of the invention attached.

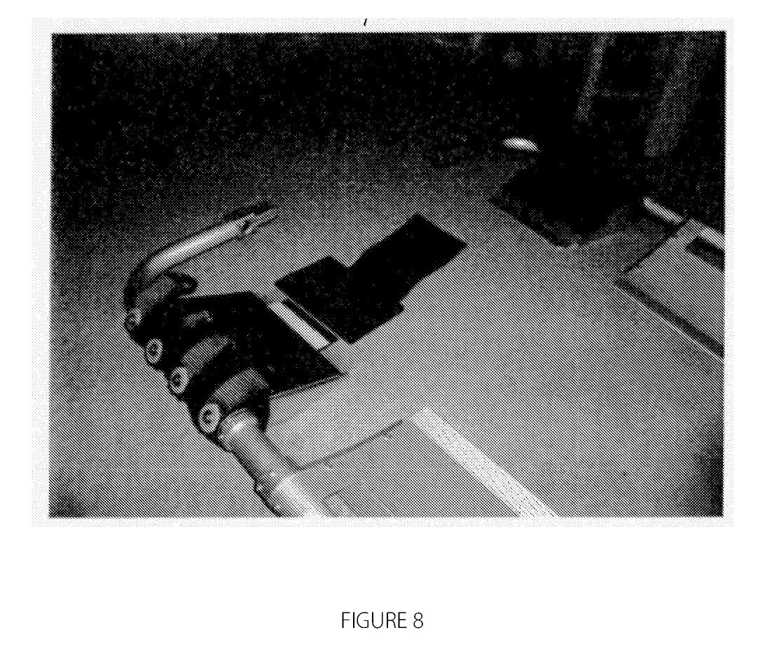

FIG. 8 shows the upper side of a second type of stretcher (a scoop stretcher with sloped head rest) with two base supports of the invention attached and a head pad of the invention between the stretcher sides.

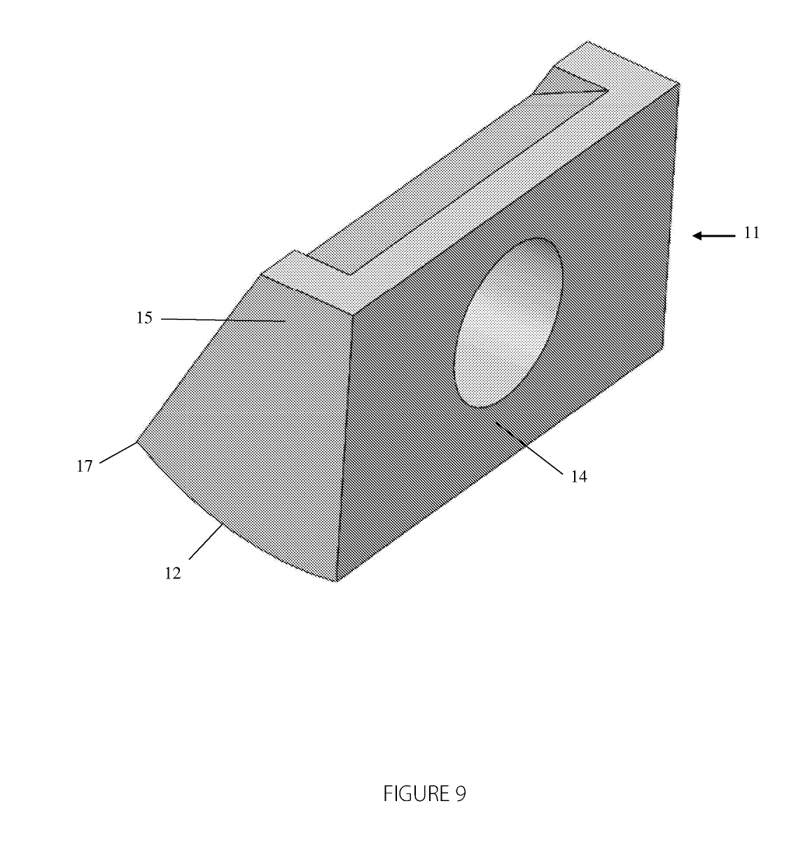

FIG. 9 shows a head block of the invention, looking down from above and showing the side that would be used to support a person's head.

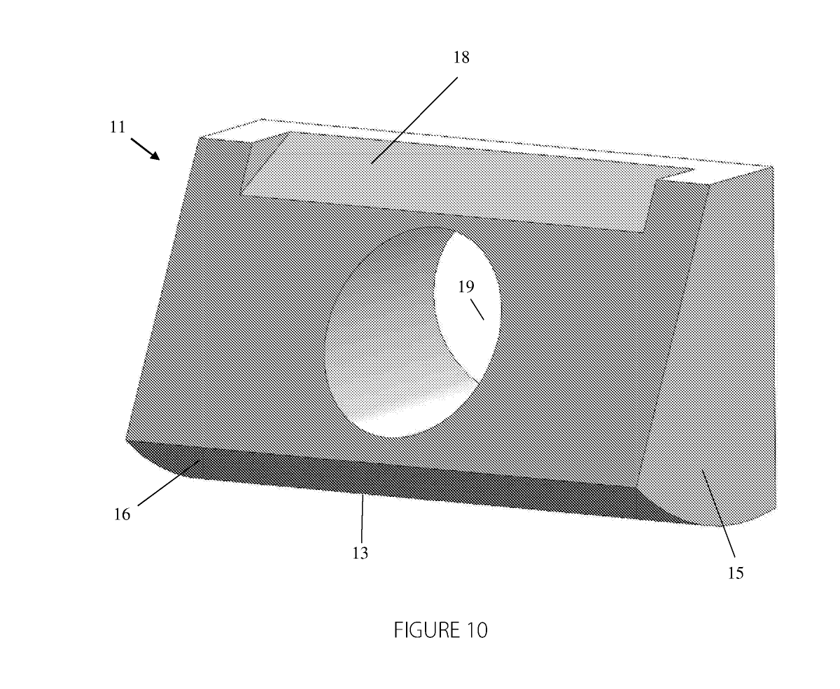

FIG. 10 shows a head block of FIG. 9 from the back, showing the surface of the curved base.

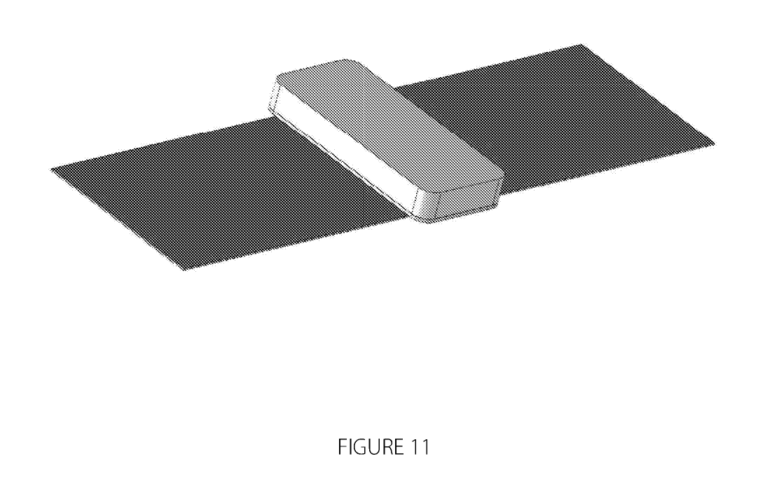

FIG. 11 shows a head pad of the invention with material sides which may be used to attach it to one or more base supports.

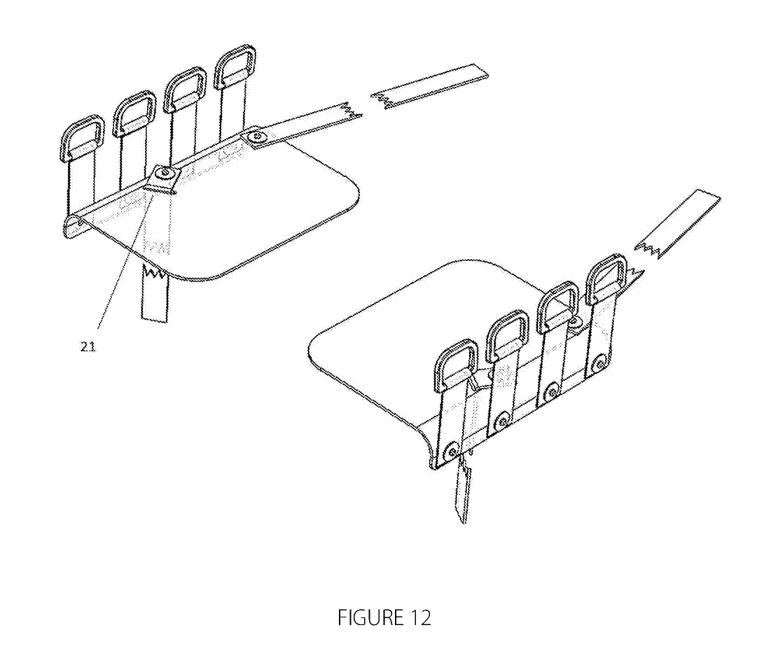

FIG. 12 shows a pair of base supports of the invention which have a hole through which an attachment strap has been passed.

Turning first to FIGS. 1 to 5, the base support 1 of the invention comprises a base plate 2 comprising a sandblasted aluminium framework 20 (see FIGS. 2, 3 and 5). The top of the plate 2 has a layer 3 of the loop component of hook and loop material glued on to the framework. In this instance, the loop material is black. The plate 2 is generally rectangular and continues at one of the long edges into a curved extension 4. The extension 4 can loop around a handle of a stretcher (see FIGS. 6 to 8). The dimensions (looking top down at the plate, e.g. as in FIG. 4) are 160 mm by 190 mm.

On the outside edge of the extension 4, four head strap connectors, 5a to 5d, are attached to the upper side of the plate (the side which, in use, would be on top as opposed to adjacent to the stretcher). The head restraining strap connectors are attached by rivets to the plate 2. The head strap connectors are made principally of webbing material and terminate in D-rings 6a to 6d. The D-rings facilitate attachment of the connectors to head straps for use with head immobilisers (not shown).

Opposite each other along the short edges of the flat section of the generally rectangular plate 2 there are two attachment straps 7a and 7battached to the upper side of the plate. The attachment straps are made of 2 cm wide lengths of hook and loop fastener material stitched together back to back. They are attached to the plate 2 by rivets. After looping through handles/around edges on a stretcher, the distal ends 8a and 8b of the attachment straps (those furthest from the plate attachment rivets) can thus be adhered to the loop component 3.

As seen in FIGS. 2 and 5, the underside of the base plate also has areas 9a and 9b on which there is glued on loop component of hook and loop fastener. These protect parts of the stretcher from wear and tear. The underside loop material is continued round plate edges from the upper side.

As indicated in FIGS. 3 to 8, the base supports are intended to be used in pairs, with one being fitted on each side of a stretcher (see FIGS. 6 to 8).

Accordingly, in use, two of the base supports are attached to a stretcher (there to be one either side of a person's head) as previously described using the attachment straps. This is preferably before a patient is transferred to the stretcher, but could be after. A head block, having an area with a layer of the hook component of hook and loop fastener can then be adhered to the loop component 3 on top of the plate 2 of each of the base supports. A head block could also be adhered to the reverse loop sides of the attachment straps, further securing the base support attachment straps. Head straps, including chin straps, which are known to medical personnel, can then be attached as appropriate to one or more of the D rings 6a to 6d, as desired, and used to secure the head blocks and the patient's head.

In the example base support of the invention depicted in FIG. 12 the base support has a hole 21. The hole is in the form of a slit/slot and it can be used to allow an attachment strap to be passed from one side of the plate to the other such that attachment to a stretcher may be facilitated.

FIGS. 4 and 5 show that a head pad 10 can be placed between a pair of base supports. A person's head can rest on the head pad for greater comfort. As indicated in FIGS. 8 and 11, a head pad of the invention may have sides of fastener component for attachment to the base supports. (Optionally using fastener component on the underside of the base supports.) A base support embodiment may optionally have a shaped corner (e.g. as visible in FIG. 5), for example to better fit with a particular stretcher, but this is clearly not obligatory.

A head block 11 of the present invention is seen in FIGS. 9 and 10. As can be seen, the head block 11 is essentially a ‘quadrilateral’ prism on its side with one rounded face 12 along the prism length forming the bottom 13 of the block; the left and right 15 ‘quadrilaterals’ having an identical rounded side. In use, the block sits on the curved base/bottom 13. As is illustrated in FIG. 10, the base 13 is covered with a layer of hook fastener material 16.

In use, the head block is attached to a base support by means of the hook material 16. The curved base is rolled/pressed onto the base plate and the head block is positioned so that the side which may sit next to a person's head is generally vertical. Depending on the nature of the stretcher being used, the curved base will contact the base support to differing degrees.

The head block has a greatest height of 140 mm, a length of 240 mm and an upper width of 50 mm. Taken from the raised end 17 of the arc the block height is 115 mm and the width is 110 mm. The arc height is therefore 25 mm. It will be apparent that the long straight side 14 which forms a right angle with the narrow top of the block would be that which sits adjacent to a patient's head.

On the upper end of the block there is a cut-out portion 18. The cut-out portion 18 is for ease of application of head straps. It will be apparent that head blocks of the invention need not necessarily have such a cut-out portion. A cylindrical hole 19 extends through the block between the side that would be adjacent to a person's head and the opposite side. This is to allow some comfort to a patient's ear, facilitates hearing and permits a visual inspection by medical staff. It may also be used to grip the block. Again, such a feature may be considered optional.

1. A base support for a head immobiliser system for use with a stretcher, the base support being operable to be attached to one side of a stretcher such that, in use, a head block for supporting a person's head can be placed on it;
wherein the base support comprises a rigid plate, at least one head restraining strap connector secured to and extending from the plate for connection to a head restraining strap, and at least one attachment strap secured to and extending from the plate, for attaching the base support to a stretcher, and comprising an area of a component of a hook and loop fastener;
wherein the plate comprises an area of a component of a hook and loop fastener, on its upper surface, for attachment to a complementary section of fastener component on a head block surface, and an area of a component of a hook and loop fastener for attachment to the fastener component on the at least one attachment strap;
and wherein one side of the rigid plate has an extension operable to be fitted at least partially around the edge of a stretcher.

2. A base support according to claim 1 wherein the rigid plate comprises a metal framework.

3. A base support according to claim 2 wherein the metal is aluminium.

4. A base support according to claim 1, wherein the area of fastener component for attaching to a head block and the area of fastener component for attaching to the attachment strap are the same.

5. A base support according to claim 4 wherein the area of fastener component covers substantially all of the upper side of the rigid plate.

6. A base support according to claim 1 comprising a layer of material on at least a portion of the underside of the plate for protecting a stretcher when the base support is in use.

7. A base support according to claim 6 wherein the material covers substantially all of the underside of the rigid plate.

8. A base support according to claim 6 wherein the material is a component of hook and loop fastener.

9. A base support according to claim 8 wherein the hook and loop fastener component is loop fastener.

10. A base support according to claim 1 wherein the support plate is (generally) rectangular in shape.

11. A base support according to claim 1 comprising two, three, four or five head restraining strap connectors.

12. A base support according to claim 1 wherein the one or more head strap connector(s) extend(s) from the base plate extension which, in use, runs along the outside edge of the stretcher.

13. A base support according to claim 1 comprising two attachment straps.

14. A base support according to claim 13 wherein the attachment straps extend from opposite ends of the base plate which run generally perpendicular to the rigid plate extension.

15. A base support according to claim 1 wherein the fastener component area on the one or more attachment strap(s) comprises back to back lengths of hook and loop components (one component on either face).

16. A base support according to claim 1 wherein the fastener component for attaching to a head block is loop material.

17. A base support according to claim 1 wherein the one or more head restraining strap connector(s) and/or the one or more attachment strap(s) is/are secured to the upper side of the plate.

18. A base support according to claim 1 wherein the one or more head restraining strap connector(s) and/or the one or more attachment strap(s) is/are secured to the plate by stud fastenings.

19. A base support according to claim 1 comprising at least one hole for enabling passage of an attachment strap.

20. A set of head immobiliser base supports for use with a stretcher, the set comprising two base supports according to claim 1 for use on opposite sides of a stretcher.

21-28. (canceled)
